(19) United States
(12) Patent Application Publication
(10) Pub. No.: US 2022/0401635 A1
(43) Pub. Date: Dec. 22, 2022

(54) APPARATUS AND METHOD FOR DETECTING THE APPROXIMATE NUMBER OF LEUKOCYTES IN A DIALYSATE AFTER THE USE THEREOF IN PERITONEAL DIALYSIS

(71) Applicants: Franz Ferdiand Becker, Rodgau (DE); Ulrich Paul Hinkel, Weimar (DE)

(72) Inventors: Franz Ferdiand Becker, Rodgau (DE); Ulrich Paul Hinkel, Weimar (DE)

(21) Appl. No.: 17/842,358

(22) Filed: Jun. 16, 2022

(30) Foreign Application Priority Data
Jun. 17, 2021 (DE) 102021115737.4

(51) Int. Cl. A61M 1/28; G01N 15/06; G01N 33/49

(52) U.S. Cl. CPC A61M 1/28; G01N 15/06; G01N 33/49; G01N 2015/0065

(57) ABSTRACT

An apparatus for detecting the approximate number of leukocytes in a dialysate after the use thereof in peritoneal dialysis and for the analysis thereof, where the apparatus includes a housing having a through-opening for inserting a light-permeable tube and having an integrated tube clamping device, at least one laser light source directed onto the tube, a light detector device having a signal evaluation device, a display, and a power supply for the light source, the light detector device having the light signal evaluation device, and the display. The laser light source and the light detector device are arranged at an angle to one another such that the light detector device only detects the scattered radiation reflected from scattering surfaces of solid particles and in that an LED light source is also arranged, which directs the light thereof onto the light detector device which measures the transmitted light intensity and, after detecting turbidity of the dialysate, increases the value of the detected scattered radiation accordingly.

The invention relates to an apparatus and to a method for detecting the approximate number of leukocytes and a possible increase in the number of leukocytes in a dialysate after the use thereof in peritoneal dialysis and for analysing the measured values.

Dialysis is a medical procedure that removes harmful substances from the blood. Dialysis is mainly used for acute or chronic kidney dysfunction. The present invention relates to peritoneal dialysis. With this type of dialysis, the blood purification process takes place via the peritoneum, in that a cleaning solution (dialysate) is introduced into the abdominal cavity via a catheter and drained again after a residence time of around four to five hours or longer. During this time, pollutants from the body pass through the peritoneum into the dialysate.

The most feared complication of this procedure is the infiltration of bacteria into the abdominal cavity. This results in inflammation of the peritoneum (peritonitis). Said inflammation of the peritoneum is shown by an increase in cells (leukocytes) in the flushing solution that has been drained. Normally, only one to seventy leukocytes/μl is/are present in the flushing solution, although this varies from person to person. If the number of initial leukocytes increases by more than 100 leukocytes/μl, this can indicate an early stage of peritonitis.

With 100 to approx. 500 leukocytes/μl, the bag having the drained flushing solution is still visually clear. Turbidity that is visible for a patient only occurs at approx. 1000 leukocytes/μl, which then corresponds to advanced inflammation of the peritoneum and is much too late. To this day, the patient who performs peritoneal dialysis at home has to recognise the turbidity of the dialysate himself, with the result that it is too late, and the patient is almost always admitted to a specialist clinic. In cases discovered too late, the number of leukocytes is often 20,000 to 30,000 leukocytes/μl. However, a concentration of 1,000 to 2,000 leukocytes/μl can already result in irreversible damage to the peritoneum.

DE 10 2017 008 012 A1 discloses an apparatus for detecting turbidity in an aqueous flushing solution having the features of the preamble of claim 1. In the known apparatus, a laser light source is arranged such that the light thereof passes through the tube and is directed onto the opposite detector which can detect turbidity of the liquid flowing through the tube. The amount of light detected provides information about turbidity or luminescence of the liquid and is analysed by an evaluation device that displays the measured values determined in this way. Leukocytes, for example, can be identified by adding an appropriate indicator to the liquid.

U.S. Pat. No. 8,777,891 B2 discloses an apparatus and a method for detecting an early stage of incipient peritonitis, in which apparatus a light detector device detects only the scattered radiation reflected from scattering surfaces of leukocytes, in that a laser light source is arranged parallel to the light detector device and directs the beam of the laser light source in the longitudinal direction through the tube.

The present invention is based on the object of specifying an apparatus and a method for detecting the approximate number of leukocytes in a dialysate after it has been used in peritoneal dialysis, with which apparatus or method an increase of about ten leukocytes/μl can be detected and individually attributed to an incipient peritonitis, so that a severe course can already be prevented in the development phase, and an antibiotic can be used at an early stage.

This object is achieved according to the invention by the features of claims 1 and 9.

Advantageous developments of the invention are characterised in the dependent claims.

The apparatus according to the invention contains a housing having a through-opening for inserting a light-permeable tube through which the dialysate to be examined flows, the tube being able to be fixed in the housing with a tube clamping device, at least one laser light source directed onto the tube, a light detector device having a signal evaluation device, a display for showing the determined values, and a power supply for the light source, the light detector device, and the display, the laser light source and the light detector device being arranged at an angle to one another such that the light detector device only detects the scattered radiation reflected from the scattering surfaces of solid particles. The drained dialysate contains mainly or exclusively leukocytes, so that the scattered radiation is suitable for identifying the approximate number of leukocytes. It was found that the series of measurements are in the lower concentration range of 0 to 500 leukocytes/μl and come very close to corresponding laboratory analyses.

When a patient receives the apparatus according to the invention, the initial values of the scattered radiation are first detected. If the initial value is around 5 leukocytes/μl and an increase to a maximum of 20 leukocytes/μl is detected in the course of further tests over a period of around two weeks, a further increase to 50 leukocytes/μl can still be classified as harmless. A sudden increase to 100 leukocytes/μl, on the other hand, would be an alarm signal for progressive inflammation, so that diagnostic and therapeutic measures must be taken. If the initial values are 50 to 70 leukocytes/μl in another patient, an increase to 100 leukocytes/μl is not a sign of inflammation; instead, this is only the case at a significantly higher value. The critical increase in the number of detected leukocytes must be determined individually for each user of the apparatus.

It is advantageously provided that the light detector device is a camera module having integrated signal processing and having a camera lens. The camera module can be a colour camera sensor.

The laser light source is advantageously controllable and can be provided with beam-shaping optics.

The at least one laser light source can be a red laser diode.

In addition, an LED light source can be arranged, which directs the light thereof onto the light detector device so that it measures the transmitted light intensity of the dialysate. As already mentioned above, the dialysate flowing out is visually clear when there are less than about 1,000 leukocytes/μl in the dialysate, after which a visible turbidity occurs. However, said turbidity can result in the degree of brightness of the scattered radiation being reduced, so that the determined value of the scattered radiation indicates a number of leukocytes which is too low. Depending on the degree of turbidity, the approximate number of leukocytes should be increased compared to the measured scattered light.

By means of the transmission measurement, in contrast to the scattered radiation measurement, it is possible to analyse the nature and the spectrum of the outflow liquid and to determine whether the ongoing peritoneal dialysis should be interrupted if the peritoneum is attacked and worn down by the dialysate, which can be derived by the colour of the dialysate. In order to carry out such a transmission measurement, the light from the LED light source can advantageously be alternately red, green, or blue.

All components are expediently arranged inside the housing.

The method according to the invention provides for the provision of the apparatus described above, the dialysate flowing through the tube preferably being illuminated daily at several intervals in order to detect a possible increase in the number of leukocytes. The measuring time can be approx. five seconds. It is advantageously proposed that the brightness value is measured without internal lighting at the beginning of a series of measurements. In this way, the influence of the brightness of the surroundings of the housing can be recognised and eliminated.

Advantageously, the dialysate flowing out through a tube after peritoneal dialysis is illuminated in the apparatus according to the invention. However, the dialysate can also be collected in a bag and only then, i.e. uncoupled from the patient, be removed through a tube that is inserted into the housing of the apparatus.

The apparatus according to the invention can be equipped with a telemedical connection option by means of which a notification is automatically transmitted to a treating doctor if a critical increase in leukocytes is detected.

Further details of the invention result from the following description of a preferred embodiment and from the drawings, in which:

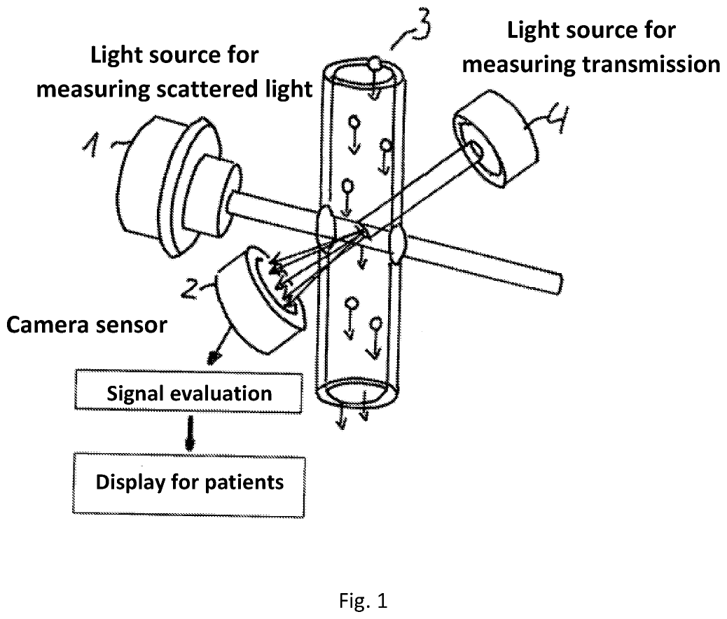

FIG. 1 shows the measuring principle of leukocyte determination in a schematic representation;

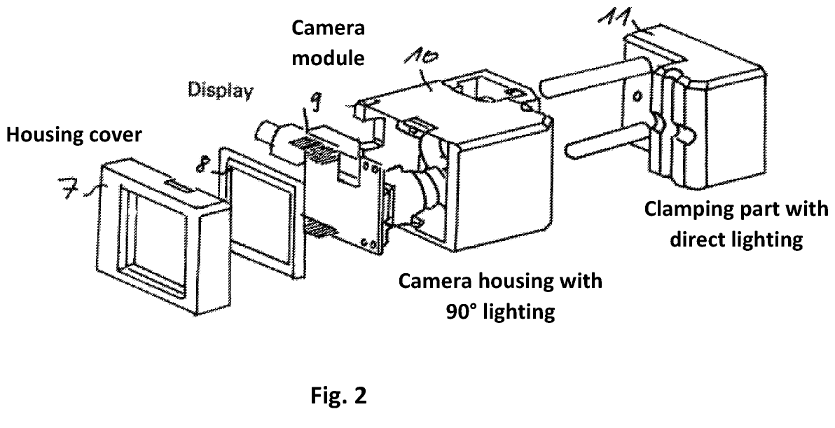

FIG. 2 shows an embodiment of the measuring device according to the invention in an exploded view.

In FIG. 1, a light laser source 1 is arranged at an angle of approximately 30° to 90° relative to the central axis of a camera sensor 2 next to a transparent tube 3 through which the dialysate to be examined flows. An LED light source 4 is directly directed at the camera sensor 2 for a transmission light measurement. The camera sensor 3 is connected to a signal evaluation system 5 and to a display device 6 for patients.

The embodiment of the measuring device according to FIG. 2 contains a housing cover 7, a display 8, a camera module 9, a camera housing 10 with 90° lighting, and a clamping part 11 with direct lighting.

1. An apparatus for detecting the approximate number of leukocytes in a dialysate after the use thereof in peritoneal dialysis and for the analysis thereof, comprising:
a housing having a through-opening for inserting a light-permeable tube and having an integrated tube clamping device,
at least one laser light source directed onto the tube,
a light detector device having a signal evaluation device,
a display, and
a power supply for the light source, the light detector device having the light signal evaluation device, and the display, wherein:
the laser light source and the light detector device are arranged at an angle to one another such that the light detector device only detects the scattered radiation reflected from scattering surfaces of solid particles, and
in that an LED light source is also arranged, which directs the light thereof onto the light detector device which measures the transmitted light intensity and, after detecting turbidity of the dialysate, increases the value of the detected scattered radiation accordingly.

2. The apparatus according to claim 1, wherein the light detector device is a camera module having integrated signal processing and having a camera lens.

3. The apparatus according to claim 2, wherein the camera module is a colour camera sensor.

4. The apparatus according to any of claim 1, wherein the at least one laser light source can be controlled with beam-shaping optics.

5. The apparatus according to claim 1, wherein the at least one laser light source is a red laser diode.

6. The apparatus according to claim 1, wherein the light of the LED light source is green.

7. The apparatus according to claim 1, wherein the light of the LED light source is alternately red, green, or blue.

8. The apparatus according to claim 1, wherein all components are arranged in the housing.

9. A method for detecting the approximate number of leukocytes in a dialysate after the use thereof in peritoneal dialysis and for the analysis thereof, the method comprising providing an apparatus according to claim 1 and illuminating the dialysate flowing through the tube daily at several intervals.

10. The method according to claim 9, wherein the measuring time is about 5 seconds.

11. The method according to claim 9, wherein the brightness value is measured without internal lighting at the beginning of a series of measurements.

12. The method according to claim 9, further comprising illuminating the dialysate flowing out after peritoneal dialysis.
